(19) United States
(12) Patent Application Publication
(10) Pub. No.: US 2008/0300478 A1
(43) Pub. Date: Dec. 4, 2008

(54) SYSTEM AND METHOD FOR DISPLAYING REAL-TIME STATE OF IMAGED ANATOMY DURING A SURGICAL PROCEDURE

(75) Inventors: Joel Frederick Zuhars, Haverhill, MA (US); Thomas C. Kienzle, III, Lake Forest, IL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/755,122

(22) Filed: May 30, 2007

(51) Int. Cl. A61B 5/00

(52) U.S. Cl. 600/407

(57) ABSTRACT

A system and method for displaying surgical instruments accurately, in both time and space, within both slice and volumetric medical images of organs that may deform in a predictable manner over time due to an ongoing body activity. The system and method comprising using a gating signal associated with an ongoing body activity to determine which image in a sequence of acquired images best represents the state of the imaged anatomy at a given point in time and accurately displaying navigated surgical instruments within that image at that point in time.

### FIELD OF THE INVENTION

This disclosure relates generally to image-guided surgery systems (or surgical navigation systems), and in particular to systems and methods for displaying the real-time state of imaged anatomy, and accurately tracking and displaying surgical instruments during a surgical procedure.

### BACKGROUND OF THE INVENTION

Image-guided surgery systems track the precise location of surgical instruments in relation to multidimensional images of a patient's anatomy. Additionally, image-guided surgery systems use visualization tools to provide the surgeon with co-registered views of these surgical instruments with the patient's anatomy. The multidimensional images of a patient's anatomy may include computed tomography (CT) imaging data, magnetic resonance (MR) imaging data, positron emission tomography (PET) imaging data, ultrasound imaging data, X-ray imaging data, or any other suitable imaging data, as well as any combinations thereof.

Several surgical procedures require very precise planning for placement of surgical instruments that are internal to the body and difficult to view during the procedure. This is especially true when dealing with internal organs or anatomy that may deform in a predictable manner over time due to ongoing body activity, such as breathing and the beating of the heart.

Registration of 3D image datasets (CT, MR, PET, ultrasound, etc.) to a known reference frame can be a difficult problem in the operating room. The initial registration is typically defined by identifying common fiducial points within a region of interest between a 3D image dataset and a set of 2D or 3D fluoroscopic images. The previously acquired 3D image dataset defines a 3D rectilinear coordinate system, by virtue of their precision scan formation or the spatial mathematics of their reconstruction algorithms. However, it may be necessary to correlate 2D or 3D fluoroscopic images and anatomical features with features in a previously acquired 3D image dataset and with external coordinates of surgical instruments being used. As mentioned above, this is often accomplished by providing fiducials, or externally visible trackable markers that may be imaged, identifying the fiducials or markers on various images, and thus identifying a common set of coordinate registration points on the various images that may be tracked using a tracking system. Instead of using fiducials, tracking systems may employ an initialization process wherein the surgeon touches a number of points on a patient's anatomy in order to define an external coordinate system in relation to the patient's anatomy and to initiate tracking. In addition, image based registration algorithms can simplify the surgical workflow by using images that are available during the procedure without requiring direct contact with rigid patient landmarks.

The imaging and tracking accuracy of surgical instruments is impaired by a difference in the representation of a patient's anatomy in static images under dynamic circumstances, such as when the patient's anatomy deforms due to an ongoing body activity.

Therefore, it would be desirable to provide a system and method for tracking and displaying surgical instruments accurately in real-time, in both time and space, within both slice and volumetric medical images of organs that may deform in a predictable manner over time due to ongoing body activity.

### SUMMARY OF THE INVENTION

The above-mentioned shortcomings, disadvantages and problems are addressed herein which will be understood by reading and understanding the following specification.

In an embodiment, a method of displaying real-time state of imaged anatomy during a surgical procedure comprising the steps of attaching a measurement device to a patient to measure and ongoing body activity and generating a gating signal corresponding to the ongoing body activity; establishing a navigation reference frame around the patient in a surgical field of interest; acquiring a time series of images of a patient's anatomy in the surgical field of interest in sync with the gating signal; determining an image position for each image in the time series in relation to the navigation reference frame; determining a position for at least one navigated surgical instrument used during the surgical procedure in relation to the navigation reference frame; and displaying the time series of images along with the at least one navigated surgical instrument used during the surgical procedure superimposed on the time series of images showing the real-time state of the imaged anatomy and the accurate positions of the at least one navigated surgical instrument within the real-time state of imaged anatomy.

In an embodiment, a method of displaying real-time state of imaged anatomy during a surgical procedure comprising the steps of attaching a measurement device to a patient to measure and ongoing body activity and generating a gating signal corresponding to the ongoing body activity; acquiring a time series of pre-operative 3D images of a patient's anatomy in a surgical field of interest in sync with the gating signal; establishing a navigation reference frame around the patient in the surgical field of interest; acquiring a time series of intraoperative 2D images of the patient's anatomy in the surgical field of interest in sync with the gating signal; reconstructing a time series of intraoperative 3D images from the time series of intraoperative 2D images; registering the time series of pre-operative 3D images with the time series of intraoperative 3D images; and displaying the time series of pre-operative 3D images along with at least one navigated surgical instrument used during the surgical procedure superimposed on the time series of pre-operative 3D images showing the real-time state of the imaged anatomy and the accurate positions of the at least one navigated surgical instrument within the real-time state of imaged anatomy.

In an embodiment, a method of displaying real-time state of imaged anatomy during a surgical procedure comprising the steps of attaching an EKG measurement device to a patient to measure the EKG of a patient and generating a gating signal corresponding to the measured EKG; acquiring a time sequence of pre-operative 3D CT volumetric images of a patient's anatomy in a surgical field of interest in sync with the gating signal; establishing a navigation reference frame around the patient in the surgical field of interest; acquiring a series of intraoperative 2D fluoroscopic images of the patient's anatomy in the surgical field of interest taken at various intervals while an imaging apparatus rotates around the patient; recording the EKG measurement and an image position within the navigation reference frame for each image in the series of intraoperative 2D fluoroscopic images; reconstructing a series of intraoperative 3D fluoroscopic volumetric images from the series of intraoperative 2D fluoroscopic images that have approximately the same EKG measurement; registering each intraoperative 3D fluoroscopic volumetric image in the time series with the navigation reference frame; registering each intraoperative 3D fluoroscopic volumetric image in the series with the corresponding pre-operative 3D CT volumetric image in the time sequence; and displaying the time sequence of pre-operative 3D CT volumetric images along with at least one navigated surgical instrument used during the surgical procedure superimposed on the time sequence of pre-operative 3D CT volumetric images showing the real-time state of the patient's imaged anatomy and the accurate positions of the at least one navigated surgical instrument within the real-time state of the patient's imaged anatomy.

In an embodiment, a method of displaying real-time state of imaged anatomy during a surgical procedure comprising the steps of attaching a respiratory cycle measurement device to a patient to measure the respiratory cycle of the patient and generating a gating signal corresponding to the measured respiratory cycle; establishing a navigation reference frame around the patient in a surgical field of interest; acquiring a time series of 2D fluoroscopic images of a patient's anatomy in the surgical field of interest in sync with the gating signal; recording the respiratory cycle measurement and an image position within the navigation reference frame for each image in the time series of 2D fluoroscopic images; and displaying the time series of 2D fluoroscopic images along with navigated surgical instruments superimposed on the time series of 2D fluoroscopic images showing the real-time state of the patient's imaged anatomy and the accurate positions of the navigated surgical instruments within the real-time state of the patient's imaged anatomy.

In an embodiment, an image-guided surgery system comprising a measurement device coupled to a patient for measuring an ongoing body activity of the patient and generating a gating signal corresponding to the ongoing body activity measurement; a plurality of tracking elements coupled to a navigation apparatus, wherein the navigation apparatus includes at least one processor; at least one imaging apparatus coupled to the navigation apparatus configured for imaging a patient's anatomy that deforms and changes position in relation to the ongoing body activity; and at least one display coupled to the a navigation apparatus and at least one imaging apparatus configured for displaying 2D slice and 3D volumetric images of the patient's anatomy that deforms and changes position in relation to the ongoing body activity and displaying an accurate position of at least one navigated surgical instrument within the 2D slice and 3D volumetric images in real-time.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an exemplary embodiment of an image-guided surgery system;

FIG. 2 is a block diagram of an exemplary embodiment of an image-guided surgery system;

FIG. 3 is a flow diagram of an exemplary embodiment of a method for displaying the real-time state of imaged anatomy, and accurately tracking and displaying surgical instruments within the imaged anatomy during a surgical procedure;

FIG. 4 is a flow diagram of an exemplary embodiment of a method for displaying the real-time state of imaged anatomy, and accurately tracking and displaying surgical instruments within the imaged anatomy during a surgical procedure;

FIG. 5 is a more detailed flow diagram of the method of FIG. 4;

FIG. 6 is a flow diagram of an exemplary embodiment of a method for displaying the real-time state of imaged anatomy, and accurately tracking and displaying surgical instruments within the imaged anatomy during a surgical procedure using an EKG signal as a gating signal; and

FIG. 7 is a flow diagram of an exemplary embodiment of a method for displaying the real-time state of imaged anatomy, and accurately tracking and displaying surgical instruments within the imaged anatomy during a surgical procedure using a respiratory signal as a gating signal.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the appended claims.

In various embodiments, a system and method for displaying the real-time state of imaged anatomy, and accurately tracking and displaying surgical instruments during a surgical procedure is disclosed. The disclosure provides a system and method that combines image-guided surgery (e.g., a surgical navigation) with time sequenced 2D images and/or 3D images by using one or more measures of an ongoing body activity, such as an EKG measurement or a respiratory cycle measurement, as a gating signal to determine which image in a sequence best represents the state of the imaged anatomy at a given point in time, and then displaying a navigated instrument or implant within that image at the given point in time.

The disclosure is explained with reference to selected surgical procedures using selected gating signals. However, it should be appreciated that the disclosure need not be limited to any surgical procedures or any gating signals. The systems and methods described may be used in any surgical procedure, where a patient's anatomy deforms due to ongoing body activity. The gating signal may be associated with a body activity, and correlated with the behavior of the patient's anatomy. For example, a surgical procedure related to the heart may be linked with an EKG signal as the gating signal, and a surgical procedure related to the liver may be linked with a respiratory cycle signal as the gating signal.

In surgical procedures, access to the body is obtained through one or more small percutaneous incisions or one larger incision in the body. Surgical instruments are inserted through these openings and directed to a region of interest within the body. Direction of the surgical instruments or implants through the body is facilitated by navigation technology wherein the real-time location of a surgical instrument or implant is measured and virtually superimposed on an image of the region of interest. The image may be a pre-acquired image, or an image obtained in near real-time or real-time using known imaging technologies such as computed tomography (CT), magnetic resonance (MR), positron emission tomography (PET), ultrasound, X-ray, or any other suitable imaging technology, as well as any combinations thereof.

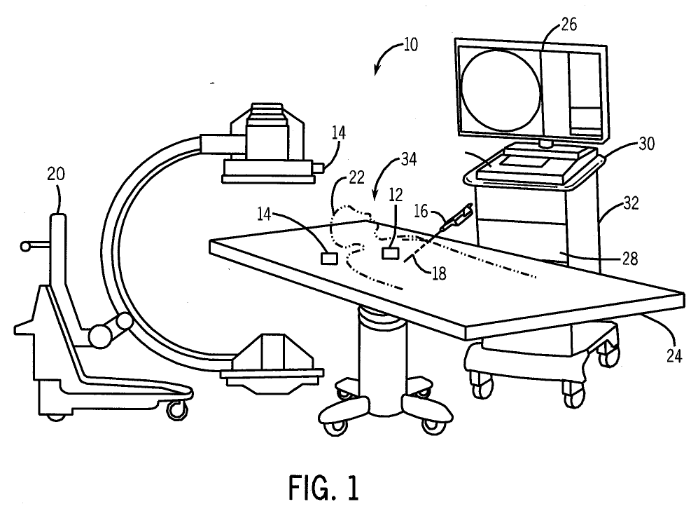

Referring now to FIG. 1, an image-guided surgery system (e.g., a surgical navigation system), designated generally by reference numeral 10 is illustrated. The system 10 includes a plurality of tracking elements 12, 14, 16 positioned proximate to a surgical field of interest 34, a navigation apparatus 30 coupled to and receiving data from the plurality of tracking elements 12, 14, 16, at least one imaging apparatus 20 coupled to navigation apparatus 30 for performing imaging on a patient 22 in the surgical field of interest 34, and at least one display 26 coupled to at least one imaging apparatus 20 and navigation apparatus 30 for displaying imaging and tracking data from the image-guided surgery system. The patient 22 is shown positioned on a table 24 as an example of the setup during a surgical procedure. The navigation apparatus 30 and at least one display 26 are shown mounted on a portable cart 32 in the embodiment illustrated in FIG. 1.

The image-guided surgery system 10 also includes a measurement device 28 coupled to the patient 22, navigation apparatus 30 and at least one imaging apparatus 20 for measuring an ongoing body activity of patient 22. For example, the ongoing body activity may be breathing (respiration) or the beating of the heart, wherein patient anatomy deforms in a predictable manner over time due to the ongoing body activity. The measurement device 28 may be mounted on the portable cart 32 that includes components of the navigation apparatus 30 or may be positioned separately from the navigation apparatus 30. The measurement device 28 is configured for generating a gating signal associated with the ongoing body activity. For example, the measurement device 28 may be a respiratory cycle measurement device for measuring respiratory cycles of patient 22 or an electrocardiogram (EKG) measurement device for measuring cardiac cycles of patient 22. The respiratory cycle measurement device provides a gating signal associated with the changes in respiration detected by a change in position of a first tracking element 12 attached to the patient's chest relative to a second tracking element 14 that is fixed to the table 24, at least one imaging apparatus 20 or other fixed location. The EKG measurement device provides a gating signal associated with variations in electrical potential caused by the excitation of the heart muscle and detected at the body surface by sensors.

The gating signal generated by measurement device 28 may be coupled to at least one imaging apparatus 20 and navigation apparatus 30, and used to trigger image and data acquisition. The measurement device 28 measures an ongoing body activity of patient 22 and produces a gating signal off of the ongoing body activity to be used for triggering image and data acquisition of imaging apparatus 26 and navigation apparatus 30. The at least one imaging apparatus 20 automatically acquires data for a time series of images or for images at different anatomical levels.

The plurality of tracking elements 12, 14, 16 are operative to determine the positions of a patient's anatomy and surgical instruments. A first tracking element 12 may be attached to patient 22 on the patient's anatomy that changes position due to an ongoing body activity, a second tracking element 14 may be attached to at least one imaging apparatus 20 or attached to table 24 near the patient's anatomy that changes position due to an ongoing body activity, and a third tracking element 16 attached to a surgical instrument 18 to which an implant may be attached. For example, the first tracking element 12 may be attached to the chest of patient 22 and the second tracking element 14 may be attached to table 24 near the chest of patient 22. The location of the first tracking element 12 attached to patient 22 may change based on the ongoing body activity being measured. The plurality of tracking elements 12, 14, 16 may be coupled to the navigation apparatus 30 through either a wired or wireless connection.

In an exemplary embodiment, at least one tracking element 12 or 14 may act as a navigation reference that may be attached to patient 22 or table 24 near patient 22 in the surgical field of interest 34. The navigation reference creates a navigation reference frame for the image-guided surgery system 10 around the patient's anatomy in the surgical field of interest 34. Typically, the navigation reference used by an imaged-guided surgery system 10 is registered to the patient's anatomy prior to performing image-guided surgery or surgical navigation. Registration of the navigation reference frame impacts the accuracy of a navigated surgical instrument 18 or implant in relation to a displayed image.

In an exemplary embodiment, at least one tracking element 14 may act as a positional reference that may be attached to at least one imaging apparatus 20. The positional reference assists navigation apparatus 30 in determining imaging position in relation to at least one imaging apparatus 20.

In an exemplary embodiment, at least two tracking elements 12, 14 may be used for measuring an ongoing body activity. For example, for measuring the respiration cycle of a patient, a first tracking element 12 may be attached to the chest of patient 22 and a second tracking element 14 may be attached to table 24 or attached to at least one imaging apparatus 20. The navigation apparatus 30 would then be used to measure the difference in position between the first tracking element 12 attached to the chest of patient 22 and the second tracking element 14 attached to table 24 or attached to at least one imaging apparatus 20 during patient respiration.

In an exemplary embodiment, the navigation apparatus 30 may be configured for providing positional information of images relative to surgical instruments 18 and instrument navigation coordinates representing the tracking position of surgical instruments 18 in a patient's anatomy with reference to a gating signal in real-time during a surgical procedure.

In an exemplary embodiment, the at least one imaging apparatus 20 may be a fluoroscopic imaging apparatus for use during a surgical procedure. The at least one imaging apparatus 20 may be coupled to navigation apparatus 30 through either a wired or wireless connection. A second imaging apparatus (not shown) may be used to acquire a plurality of high quality images prior to performing the surgical procedure. This second imaging apparatus may comprise CT, MR, PET, ultrasound, X-ray, or any other suitable imaging technology, as well as any combinations thereof.

In an exemplary embodiment, the at least one display 26 is configured to show the real-time position and orientation of surgical instruments and/or implants on registered images of a patient's anatomy. Graphical representations of the surgical instruments and/or implants are shown on the display. These representations may appear as line renderings, shaded geometric primitives, or realistic 3D models from computer-aided design (CAD) files. The images and instrument representations are continuously being updated in real-time by the gating signal. The at least one display 26 may be coupled to at least one imaging apparatus 20 and navigation apparatus 30 through either a wired or wireless connection.

In an exemplary embodiment, the image-guided surgery system 10 may be an electromagnetic surgical navigation system utilizing electromagnetic navigation technology. However, other tracking or navigation technologies may be utilized as well.

In an electromagnetic surgical navigation system, the plurality of tracking elements 12, 14, 16 may include electromagnetic field generators and electromagnetic sensors that allow a surgeon to continually track the position and orientation of at least one surgical instrument 18 or an implant during a surgical procedure.

The electromagnetic field generators may include at least one coil, at least one coil pair, at least one coil trio, or a coil array for generating an electromagnetic field. A current is applied from the navigation apparatus 30 to the at least one coil, at least one coil pair, at least one coil trio, or a coil array of the electromagnetic field generators to generate a magnetic field around the electromagnetic field generators. The electromagnetic sensors may include at least one coil, at least one coil pair, at least one coil trio, or a coil array for detecting the magnetic field. The electromagnetic sensors are brought into proximity with the electromagnetic field generators in the surgical field of interest 14. The magnetic field induces a voltage in the at least one coil, at least one coil pair, at least one coil trio, or a coil array of the electromagnetic sensors, detecting the magnetic field generated by the electromagnetic field generators for calculating the position and orientation of the at least one surgical instrument 18 or implant. The electromagnetic sensors may include electronics for digitizing magnetic field measurements detected by the electromagnetic sensors. It should, however, be appreciated that according to alternate embodiments the electromagnetic field generators may be electromagnetic sensors, and the electromagnetic sensors may be electromagnetic field generators.

The magnetic field measurements can be used to calculate the position and orientation of at least one surgical instrument 18 or implant according to any suitable method or system. After the magnetic field measurements are digitized using electronics, the digitized signals are transmitted from the electromagnetic sensors to a computer or processor within the navigation apparatus 30 through a navigation interface. The digitized signals may be transmitted from the electromagnetic sensors to the navigation apparatus 30 using wired or wireless communication protocols and interfaces. The digitized signals received by the navigation apparatus 30 represent magnetic field information detected by the electromagnetic sensors. The digitized signals are used to calculate position and orientation information of the at least one surgical instrument 18 or implant. The position and orientation information is used to register the location of the surgical instrument 18 or implant to acquired imaging data from at least one imaging apparatus 20. The position and orientation data is visualized on at least one display 26, showing in real-time the location of at least one surgical instrument 18 or implant on pre-acquired or real-time images from at least one imaging apparatus 20. The acquired imaging data from at least one imaging apparatus 20 may include CT imaging data, MR imaging data, PET imaging data, ultrasound imaging data, X-ray imaging data, or any other suitable imaging data, as well as any combinations thereof. In addition to the acquired imaging data from various modalities, real-time imaging data from various real-time imaging modalities may also be available.

In an exemplary embodiment, the image-guided surgery system 10 may be integrated into a single integrated imaging and navigation system with integrated instrumentation and software.

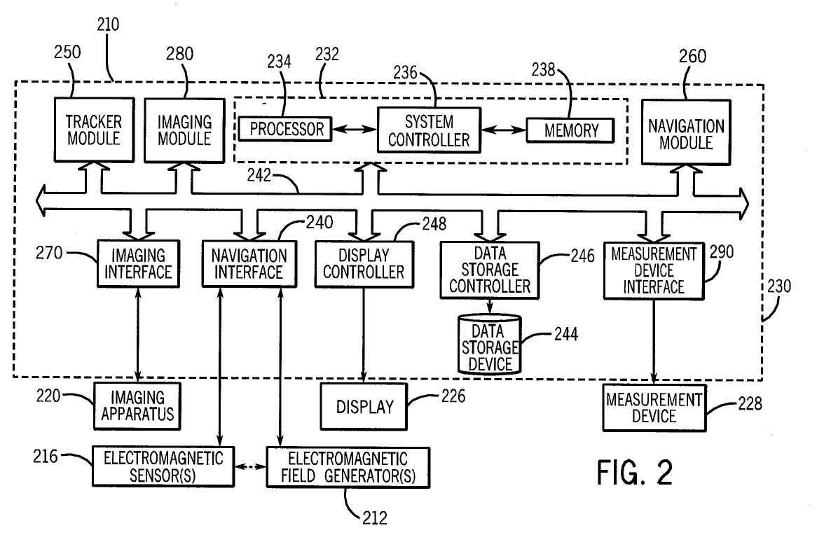

FIG. 2 is a block diagram of an exemplary embodiment of an image-guided surgery system 210 utilizing electromagnetic navigation technology. The image-guided surgery system 210 is illustrated conceptually as a collection of modules and other components that are included in a navigation apparatus 230, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for position and orientation calculations as well as dedicated processors for imaging operations and visualization operations. As a further option, the modules may be implemented using a hybrid configuration in which certain modular functions are performed using dedicated hardware, while the remaining modular functions are performed using an off-the-shelf computer. In the embodiment shown in FIG. 2, the image-guided surgery system 210 includes a computer 232 having a processor 234, a system controller 236 and memory 238. The processor 234 is programmed with integrated software for planning and performing a surgical procedure. The operations of the modules and other components of the navigation apparatus 230 may be controlled by the system controller 236.

The image-guided surgery system 210 includes a plurality of tracking elements that may be in the form of electromagnetic field generators 212 and electromagnetic sensors 216 that are coupled to a navigation interface 240. The electromagnetic field generators 212 each generate an electromagnetic field that is detected by electromagnetic field sensors 216. The navigation interface 240 receives digitized signals from electromagnetic sensors 216. The navigation interface 240 includes at least one Ethernet port. The at least one Ethernet port may be provided, for example, with an Ethernet network interface card or adapter. However, according to various alternate embodiments, the digitized signals may be transmitted from electromagnetic sensors 216 to navigation interface 240 using alternative wired or wireless communication protocols and interfaces.

The digitized signals received by navigation interface 240 represent magnetic field information from electromagnetic field generators 212 detected by electromagnetic sensors 216. In the embodiment illustrated in FIG. 2, navigation interface 240 transmits the digitized signals to a tracker module 250 over a local interface 242. The tracker module 250 calculates position and orientation information based on the received digitized signals. This position and orientation information provides a location of a surgical instrument or implant.

In an exemplary embodiment, the electromagnetic field generators 212 and electromagnetic sensors 216 may be coupled to navigation interface 240 through either a wired or wireless connection.

The tracker module 250 communicates the position and orientation information to a navigation module 260 over local interface 242. As an example, this local interface 242 is a Peripheral Component Interconnect (PCI) bus. However, according to various alternate embodiments, equivalent bus technologies may be substituted.

Upon receiving the position and orientation information, the navigation module 260 is used to register the location of the surgical instrument or implant to acquired patient imaging data. In the embodiment illustrated in FIG. 2, the acquired patient imaging data is stored on a data storage device 244. The acquired patient imaging data may include CT imaging data, MR imaging data, PET imaging data, ultrasound imaging data, X-ray imaging data, or any other suitable imaging data, as well as any combinations thereof. By way of example only, the data storage device 244 is a hard disk drive, but other suitable storage devices may be used.

Patient imaging data acquired prior to a surgical procedure may be transferred to system 210 and stored on data storage device 244. The acquired patient imaging data is loaded into memory 238 from data storage device 244. The acquired patient imaging data is retrieved from data storage device 244 by a data storage device controller 246. The navigation module 260 reads from memory 238 the acquired patient imaging data. The navigation module 260 registers the location of the surgical instrument or implant to acquired patient imaging data, and generates image data suitable to visualize the patient imaging data and a representation of the surgical instrument or implant. The imaging data is transmitted to a display controller 248 over local interface 242. The display controller 248 is used to output the imaging data to display 226.

The image-guided surgery system 210 may further include an imaging apparatus 220 coupled to an imaging interface 270 for receiving real-time imaging data. The imaging data is processed in an imaging module 280. The imaging apparatus 220 provides the ability to acquire images of a patient and display real-time imaging data in combination with position and orientation information of a surgical instrument or implant on display 226.

The image-guided surgery system 210 may further include a measurement device 228 for measuring an ongoing body activity of a patient. For example, the ongoing body activity may be breathing (respiration) or the beating of the heart, wherein patient anatomy deforms in a predictable manner over time due to the ongoing body activity. The measurement device 228 is attached to a patient with sensors to measure the ongoing body activity and coupled to a measurement device interface 290 for transmitting and receiving data. The measurement device interface is in turn coupled to the local interface 242. The measurement device 228 is configured for generating a gating signal associated with the ongoing body activity. For example, the measurement device 228 may be a respiratory cycle measurement device for measuring respiratory cycles of a patient or an EKG measurement device for measuring cardiac cycles of a patient.

While one display 226 is illustrated in the embodiment in FIG. 2, alternate embodiments may include various display configurations. Various display configurations may be used to improve operating room ergonomics, display different views, or display information to personnel at various locations.

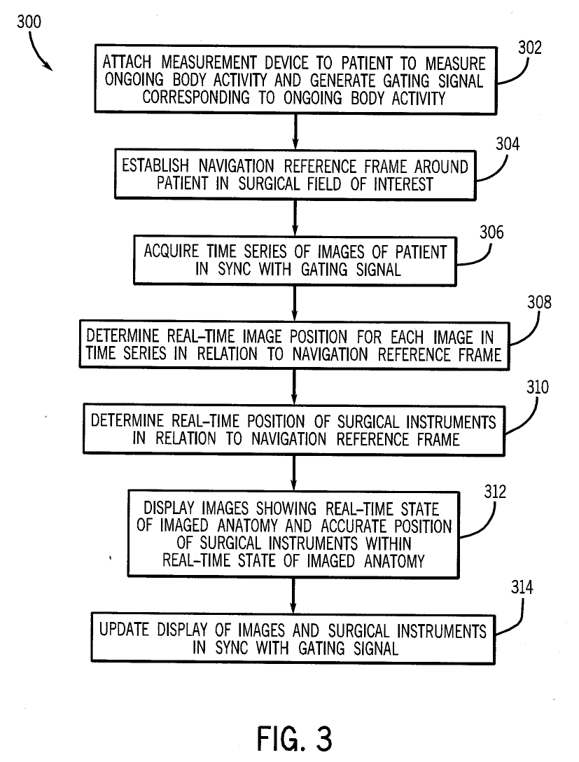

FIG. 3 is a flow diagram of an exemplary embodiment of a method 300 for displaying the real-time state of imaged anatomy, and accurately tracking and displaying surgical instruments within the imaged anatomy during a surgical procedure. The method 300 is explained with reference to a surgical procedure relating to patient anatomy that may deforms in a predictable manner over time due to an ongoing body activity, such as breathing (respiration) or the beating of the heart. At step 302, a measurement device is attached to a patient to measure an ongoing body activity and generate a gating signal corresponding to the ongoing body activity. For example, the measurement device may be an electromagnetic field generator or electromagnetic sensor to measure the respiratory cycle of a patient for liver related procedures, or a plurality of EKG sensors attached to a patient and coupled to an EKG measurement device to measure the EKG of a patient for cardiac related procedures. The measurement device generates a periodic gating signal associated with the ongoing body activity. At step 304, a navigation reference frame is established around the patient in the surgical field of interest by attaching a navigation dynamic reference device to the patient. At step 306, a time series of 2D or 3D images of the patient are obtained in the surgical field of interest. The measurement device generates a periodic gating signal associated with the ongoing body activity that is used to trigger an imaging apparatus to acquire images of the patient's anatomy in the surgical field of interest. The image acquisition is in sync with the gating signal. The gating signal is measured during the entire image scan to build each image in sequence. The images may be acquired prior to the surgical procedure (pre-operatively) and/or during the surgical procedure (intraoperatively). The images may be obtained using a CT, MR, PET, ultrasound, X-ray, or fluoroscopic imaging apparatus. The acquired images along with the corresponding gating signal are recorded and stored in a data storage device or memory of the imaging apparatus or a surgical navigation system. At step 308, an image position for each image acquired in the time series of 2D or 3D images is determined in relation to the navigation reference frame. The navigation reference frame is a transformation matrix that represents the difference between an image coordinate space and a navigation coordinate space. At step 310, a position of each surgical instrument being used on an ongoing basis is determined in relation to the navigation reference frame. At step 312, a plurality of images showing the real-time state of imaged anatomy and the accurate position of surgical instruments within the real-time state of imaged anatomy are displayed on a display of the imaging apparatus or a surgical navigation system. The display shows the real-time position and orientation of navigated surgical instruments on registered images of a patient's anatomy. Graphical representations of the navigated surgical instruments are shown on the display. The graphical representations may appear as line renderings, shaded geometric primitives, realistic 3D models from computer-aided design (CAD) files, or geometrical representations based on the instrument design drawings from manufacturers, for example. At step 314, the display of images and navigated surgical instruments is continuously updated in sync with the gating signal. The images and instrument representations are continuously being updated in real-time by the gating signal. The resulting image sequence gives the user displayed feedback, showing the anatomy deforming in real-time along with the navigated surgical instruments, thus providing a surgical benefit.

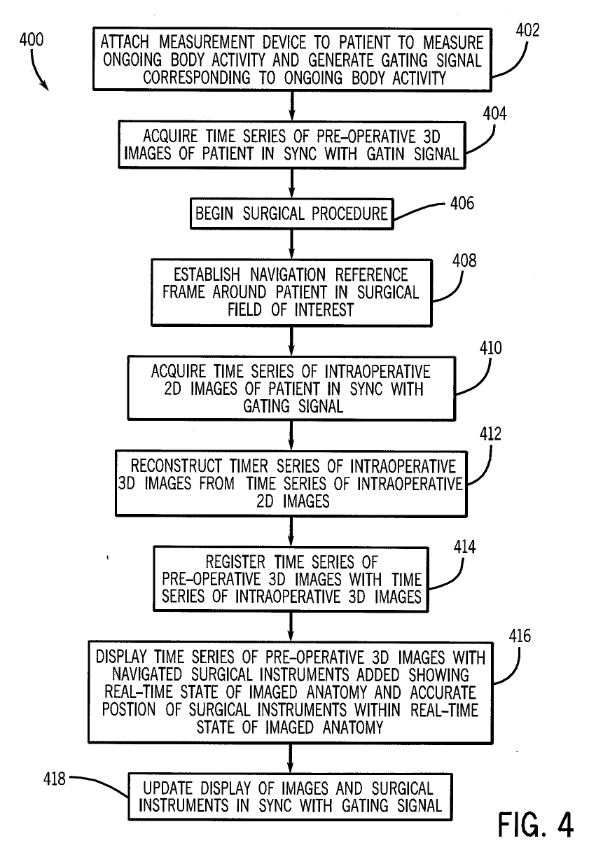

FIG. 4 is a flow diagram of an exemplary embodiment of a method 400 for displaying the real-time state of imaged anatomy, and accurately tracking and displaying surgical instruments within the imaged anatomy during a surgical procedure. The method 400 is explained with reference to a surgical procedure relating to patient anatomy that may deforms in a predictable manner over time due to an ongoing body activity, such as breathing (respiration) or the beating of the heart. At step 402, a measurement device is attached to a patient to measure an ongoing body activity and generate a gating signal corresponding to the ongoing body activity. For example, the measurement device may be an electromagnetic field generator or electromagnetic sensor to measure the respiratory cycle of a patient for liver related procedures, or a plurality of EKG sensors attached to a patient and coupled to an EKG measurement device to measure the EKG of a patient for cardiac related procedures. The measurement device generates a periodic gating signal associated with the ongoing body activity. At step 404, a time series of pre-operative 3D images of the patient are obtained in the surgical field of interest. These images are acquired pre-operatively, or prior to the surgical procedure. The measurement device generates a periodic gating signal associated with the ongoing body activity that is used to trigger an imaging apparatus to acquire images of the patient's anatomy in the surgical field of interest. The image acquisition is in sync with the gating signal. The gating signal is measured during the entire image scan to build each image in sequence. The images may be obtained using a CT, MR, PET, ultrasound, X-ray, or fluoroscopic imaging apparatus. The acquired images along with the corresponding gating signal are recorded and stored in a data storage device or memory of the imaging apparatus or a surgical navigation system. At step 406, the surgical procedure begins. At step 408, a navigation reference frame is established around the patient in the surgical field of interest by attaching a navigation dynamic reference device to the patient. At step 410, a time series of intraoperative 2D images of the patient are obtained in the surgical field of interest. These images are obtained intraoperatively, or during the surgical procedure. The measurement device generates a periodic gating signal associated with the ongoing body activity that is used to trigger an imaging apparatus to acquire images of the patient's anatomy in the surgical field of interest. The image acquisition is in sync with the gating signal. The gating signal is measured during the entire image scan to build each image in sequence. The images may be obtained using a CT, MR, PET, ultrasound, X-ray, or fluoroscopic imaging apparatus. The acquired images along with the corresponding gating signal are recorded and stored in a data storage device or memory of the imaging apparatus or a surgical navigation system. At step 412, a time series of intraoperative 3D images are reconstructed from the time series of intraoperative 2D images. An image position for each image acquired in the time series of intraoperative 3D images is determined in relation to the navigation reference frame. The navigation reference frame is a transformation matrix that represents the difference between an image coordinate space and a navigation coordinate space. At step 414, the time series of pre-operative 3D images are registered with the time series of intraoperative 3D images. An image position for each image acquired in the time series of pre-operative 3D images is determined in relation to the navigation reference frame. The method further comprises the step of determining a position for each navigated surgical instrument used during the surgical procedure in relation to the navigation reference frame. At step 416, the time series of registered pre-operative 3D images showing the real-time state of imaged anatomy and the accurate position of navigated surgical instruments used during the surgical procedure within the real-time state of imaged anatomy are displayed on a display of an intraoperative imaging apparatus or a surgical navigation system. The display shows the real-time position and orientation of navigated surgical instruments on registered images of a patient's anatomy. Graphical representations of the navigated surgical instruments are shown on the display. The graphical representations may appear as line renderings, shaded geometric primitives, realistic 3D models from computer-aided design (CAD) files, or geometrical representations based on the instrument design drawings from manufacturers, for example. At step 418, the display of images and navigated surgical instruments is continuously updated in sync with the gating signal. The images and instrument representations are continuously being updated in real-time by the gating signal. The resulting image sequence gives the user displayed feedback, showing the anatomy deforming in real-time along with the navigated surgical instruments, thus providing a surgical benefit.

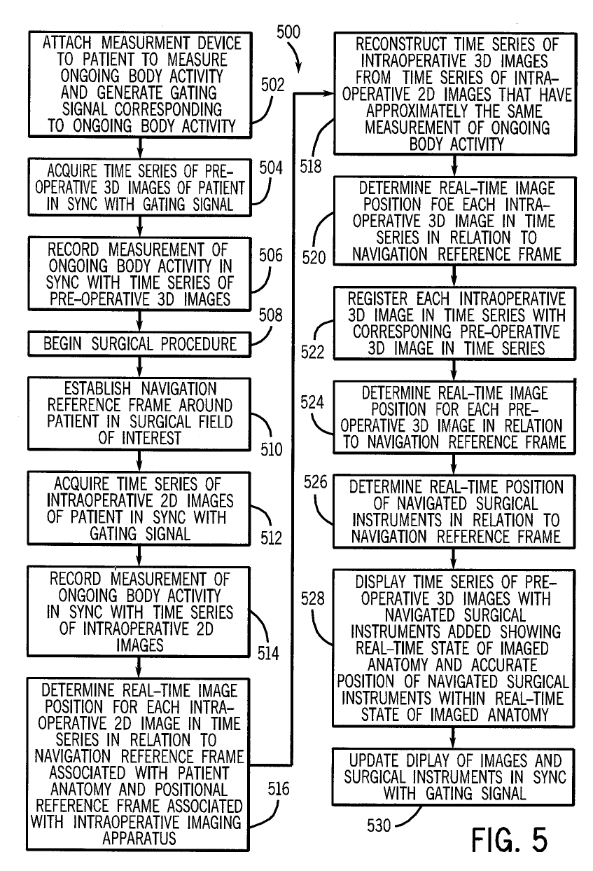

FIG. 5 is a flow diagram illustrating, in greater detail, an exemplary embodiment of a method 500 for displaying the real-time state of imaged anatomy, and accurately tracking and displaying surgical instruments within the imaged anatomy during a surgical procedure. The method 500 is explained with reference to a surgical procedure relating to patient anatomy that may deforms in a predictable manner over time due to an ongoing body activity, such as breathing (respiration) or the beating of the heart. At step 502, a measurement device is attached to a patient to measure an ongoing body activity and generate a gating signal corresponding to the ongoing body activity. For example, the measurement device may be an electromagnetic field generator or electromagnetic sensor to measure the respiratory cycle of a patient for liver related procedures, or a plurality of EKG sensors attached to a patient and coupled to an EKG measurement device to measure the EKG of a patient for cardiac related procedures. The measurement device generates a periodic gating signal associated with the ongoing body activity. At step 504, a time series of pre-operative 3D volumetric images of the patient are obtained in the surgical field of interest. The measurement device generates a periodic gating signal associated with the ongoing body activity that is used to trigger an imaging apparatus to acquire images of the patient's anatomy in the surgical field of interest. The image acquisition is in sync with the gating signal. The gating signal is measured during the entire image scan to build each image in sequence. The images may be obtained using a CT, MR, PET, ultrasound, X-ray, or fluoroscopic imaging apparatus. At step 506, the time series of pre-operative 3D volumetric images are recorded and stored along with the corresponding ongoing body activity gating signal. A representative ongoing body activity measurement for each pre-operative 3D volumetric image in the time series is recorded and stored in a data storage device or memory of the volumetric imaging apparatus or a surgical navigation system. At step 508, the surgical procedure begins. At step 510, a navigation reference frame is established around the patient in the surgical field of interest by attaching a navigation dynamic reference device to the patient. The navigation reference frame is a transformation matrix that represents the difference between an image coordinate space and a navigation coordinate space. At step 512, a time series of intraoperative 2D fluoroscopic images of the patient in the surgical field of interest are acquired using a fluoroscopic imaging apparatus. The measurement device generates a periodic gating signal associated with the ongoing body activity that is used to trigger an imaging apparatus to acquire images of the patient's anatomy in the surgical field of interest. The intraoperative 2D fluoroscopic images are acquired in sync with the gating signal. The gating signal is measured during the entire image scan to build each image in the time series. At step 514, the acquired time series of intraoperative 2D fluoroscopic images along with the corresponding gating signal are recorded and stored in a data storage device or memory of an intraoperative imaging apparatus or a surgical navigation system. At step 516, an image position for each intraoperative 2D fluoroscopic image acquired in the time series of intraoperative 2D fluoroscopic images is determined in relation to the navigation reference frame associated with the patient anatomy and a positional reference frame associated with the fluoroscopic imaging apparatus. At step 518, a time series of intraoperative 3D volumetric images are reconstructed from the time series of intraoperative 2D fluoroscopic images that have approximately the same ongoing body activity measurement. At step 520, an image position for each intraoperative 3D volumetric image in the time series is determined in relation to the navigation reference frame associated with the patient anatomy using the image positions determined for the intraoperative 2D fluoroscopic images. At step 522, each intraoperative 3D volumetric image in the time series is registered with the corresponding pre-operative 3D volumetric image in the time series. This registration maybe accomplished using an image content registration technique such as mutual information based image registration. At step 524, an image position for each pre-operative 3D volumetric image acquired in the time series is determined in relation to the navigation reference frame. At step 526, a position for each navigated surgical instrument being used in the surgical procedure is determined in relation to the navigation reference frame. At step 528, the registered pre-operative 3D images showing the real-time state of imaged anatomy and the accurate position of surgical instruments within the real-time state of imaged anatomy are displayed, in volumetric or sliced format, on a display of the imaging apparatus or a surgical navigation system. The display shows the real-time position and orientation of navigated surgical instruments on registered images of a patient's anatomy. Graphical representations of the navigated surgical instruments are shown on the display. The graphical representations may appear as line renderings, shaded geometric primitives, realistic 3D models from CAD files, or geometrical representations based on the instrument design drawings from manufacturers, for example. At step 530, the display of images and navigated surgical instrument representations is continuously updated in sync with the gating signal. The images are continuously updated in synchronization with the gating signal, and the resulting image sequence provides feedback showing the anatomy deforming in real time along with the navigated surgical instruments, thus providing a surgical benefit. The gating signal is associated with the ongoing body activity and the patient anatomy deforms in a predictable manner with reference to the gating signal.

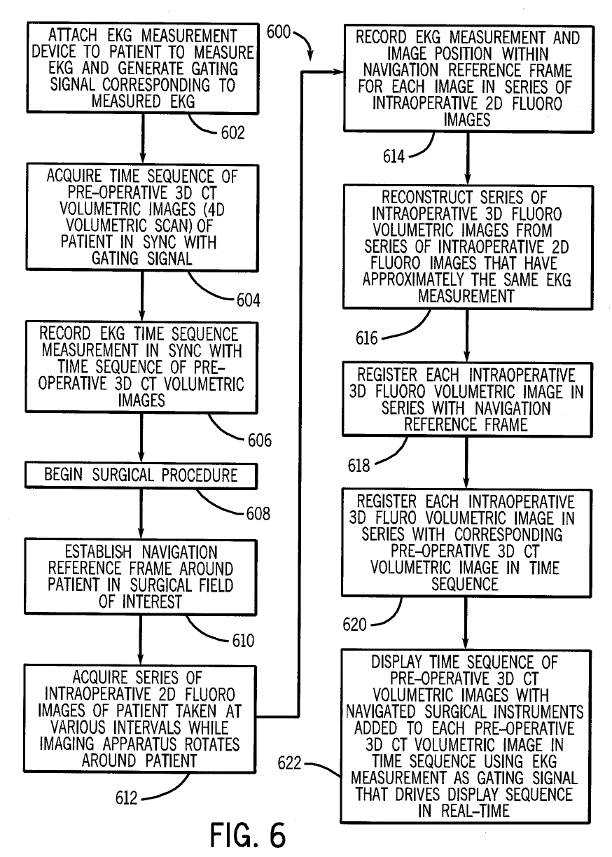

FIG. 6 is a flow diagram of an exemplary embodiment of a method 600 for displaying the real-time state of imaged anatomy, and accurately tracking and displaying surgical instruments within the imaged anatomy during a surgical procedure using an EKG signal as a gating signal. The method 600 is explained with reference to a surgical procedure relating to patient anatomy that may deform in a predictable manner over time due to the beating of the heart. At step 602, an EKG measurement device is attached to a patient to measure the EKG signal of the patient and generate a gating signal corresponding to the measured EKG signal. For example, the EKG signal measurement device may be a plurality of EKG sensors attached to a patient and coupled to an EKG measurement device to measure the EKG signal of a patient for cardiac related procedures. The EKG measurement device generates a periodic gating signal associated with the measured EKG signal of the patient. At step 604, a time sequence of pre-operative 3D volumetric images (4D volumetric scan) is acquired of the patient in the surgical field of interest. This time sequence of 3D volumetric images are acquired pre-operatively and in sync with the gating signal. For cardiac procedures, a 4D volumetric CT scan of the heart is preformed. A volumetric imaging apparatus takes multiple shots of the heart that are time sequenced to show different stages of heartbeat, for example. The images may be obtained using a CT, MR, PET, ultrasound, X-ray, or fluoroscopic imaging apparatus. At step 606, the time sequence of pre-operative 3D volumetric images are recorded and stored along with the corresponding EKG gating signal. A representative EKG measurement for each pre-operative 3D volumetric image in the time sequence is recorded and stored in a data storage device or memory of the volumetric imaging apparatus or a surgical navigation system. This involves determining a representative EKG measurement for each pre-operative 3D volumetric image in the time sequence and storing the representative EKG measurement for each pre-operative 3D volumetric image with the time sequence. The EKG time sequence data is recorded in synch with a 4D volumetric CT scan of the heart, where a representative EKG level is determined for each pre-operative 3D volumetric image in the time sequence and stored with the time sequence. At step 608, the surgical procedure begins. At step 610, a navigation reference frame is established around the patient in the surgical field of interest by attaching a navigation dynamic reference device to the patient. At step 612, a time series of intraoperative 2D fluoroscopic images of the patient in the surgical field of interest are acquired by performing a C-arm sweep of the patient. A series of images are taken at various intervals while the C-arm is rotating around the patient. This fluoroscopic CT scan is performed intraoperatively using a fluoroscopic imaging apparatus such as a C-arm. The EKG gating signal is used to trigger the fluouroscopic imaging apparatus so that the image acquisition is in synch with the gating signal. At step 614, the EKG measurement and image position within the navigation reference frame for each image in the series of intraoperative 2D fluoroscopic images is recorded and stored in a data storage device or memory of the fluoroscopic imaging apparatus or a surgical navigation system. At step 616, a series of intraoperative 3D fluoroscopic volumetric images are reconstructed from the series of intraoperative 2D fluoroscopic images that have approximately the same EKG measurement. At step 618, each intraoperative 3D fluoroscopic volumetric image in the time series is registered with the navigation reference frame. At step 620, since the intraoperative 3D fluoroscopic volumetric image quality is of significantly less quality than a traditional CT image, an image fusion technique is used to register each intraoperative 3D fluoroscopic volumetric image in the time series with the corresponding pre-operative 3D CT volumetric image in the time sequence. This registration maybe accomplished using an image content registration technique such as mutual information based image registration. This registration is used to along with the intraoperative 3D fluoroscopic volumetric image to navigation reference frame registration to display the navigated surgical instruments within the registered pre-operative 3D CT volumetric images at step 622. The time sequence of pre-operative 3D CT volumetric images, with its superior image quality, with navigated surgical instruments added to each pre-operative 3D CT volumetric image in the time sequence are displayed using the EKG measurement as the gating signal that drives the display sequence in real-time. The display shows the real-time position and orientation of navigated surgical instruments on registered images of a patient's anatomy. Graphical representations of the navigated surgical instruments are shown on the display. The graphical representations may appear as line renderings, shaded geometric primitives, realistic 3D models from CAD files, or geometrical representations based on the instrument design drawings from manufacturers, for example. The display of images and navigated surgical instruments is continuously updated in sync with the EKG gating signal.

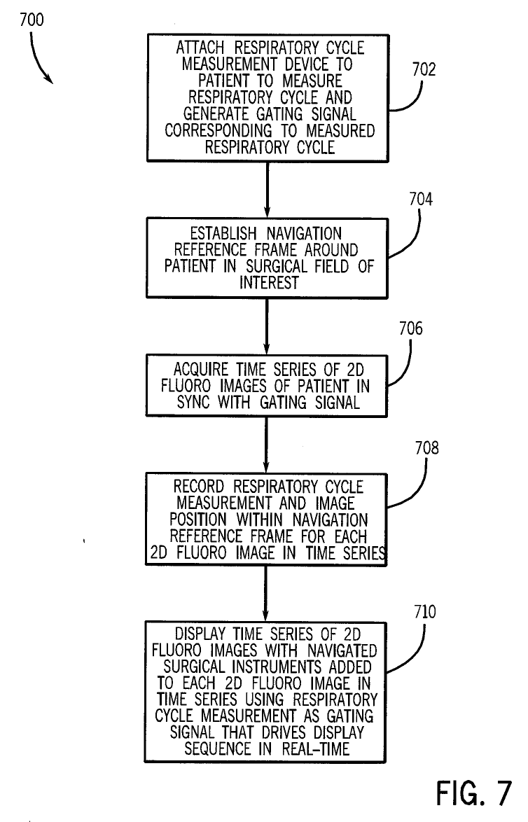

FIG. 7 is a flow diagram of an exemplary embodiment of a method 700 for displaying the real-time state of imaged anatomy, and accurately tracking and displaying surgical instruments within the imaged anatomy during a surgical procedure using a respiratory signal as a gating signal. The method 700 is explained with reference to a surgical procedure relating to patient anatomy that may deform in a predictable manner over time due to breathing (respiration). At step 702, a respiratory cycle measurement device is attached to a patient to measure the respiratory cycle of the patient and generate a gating signal corresponding to the measured respiratory cycle signal. For example, the respiratory cycle measurement device may be an electromagnetic field generator or electromagnetic sensor to measure the respiratory cycle of a patient for liver related procedures. The respiratory cycle measurement device generates a periodic gating signal associated with the measured respiratory cycle of the patient. At step 704, a navigation reference frame is established around the patient in the surgical field of interest by attaching a navigation dynamic reference device to the patient in the surgical field of interest. At step 706, a time series of 2D fluoroscopic images of the patient's anatomy in the surgical field of interest are acquired with a fluoroscopic imaging apparatus. The gating signal is used to trigger the fluouroscopic imaging apparatus. The image acquisition is in synch with the gating signal. The gating signal is measured during the entire image scan to build each image in sequence. The images are acquired during the surgical procedure. For example, this may be achieved by performing a fluoroscopic cine run (i.e., a time series of 2D fluoroscopic images are acquired with a non-moving fluoroscopic imaging apparatus) of the patient's liver or other anatomy that may deform in synchronization with patient breathing. At step 708, the respiratory cycle measurement and image position within the navigation reference frame are recorded and stored with each 2D fluoroscopic image in the time series. The respiratory cycle measurement and image position within the navigation reference frame may be recorded and stored in a data storage device or memory of the fluoroscopic imaging apparatus or a surgical navigation system. At step 710, the time series of 2D fluoroscopic images with navigated surgical instruments added to each 2D fluoroscopic image in the time series are displayed using the ongoing respiratory cycle measurement as the gating signal that drives the display sequence in real-time. The fluoroscopic imaging apparatus is removed from surgical field, and the cine run is replayed with navigated surgical instruments accurately added to each image in the time series. The re-use of the fluoroscopic cine run provides significant surgical benefit by reducing radiation dose to both the patient and surgical staff.

The benefits of this disclosure include both reduced radiation dose to the patient and surgical staff, and an improved understanding of the real-time state of the anatomy of interest, along with accurate surgical instrumentation positioning, during a surgical procedure. These benefits will contribute to improved surgical procedure outcomes.

Several embodiments are described above with reference to drawings. These drawings illustrate certain details of specific embodiments that implement the systems, methods and programs of the invention. However, the drawings should not be construed as imposing on the invention any limitations associated with features shown in the drawings. This disclosure contemplates methods, systems and program products on any machine-readable media for accomplishing its operations. As noted above, the embodiments of the may be implemented using an existing computer processor, or by a special purpose computer processor incorporated for this or another purpose or by a hardwired system.

As noted above, embodiments within the scope of the included program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments are described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the system might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The above-description of various exemplary embodiments of image-guided surgery systems and the methods have the technical effect of displaying surgical instruments accurately, in both time and space, within both slice and volumetric medical images of organs that may deform in a predictable manner over time due to an ongoing body activity.

While the invention has been described with reference to various embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A method of displaying real-time state of imaged anatomy during a surgical procedure comprising the steps of:
(a) attaching a measurement device to a patient to measure and ongoing body activity and generating a gating signal corresponding to the ongoing body activity;
(b) establishing a navigation reference frame around the patient in a surgical field of interest;
(c) acquiring a time series of images of a patient's anatomy in the surgical field of interest in sync with the gating signal;
(d) determining an image position for each image in the time series in relation to the navigation reference frame;
(e) determining a position for at least one navigated surgical instrument used during the surgical procedure in relation to the navigation reference frame; and
(f) displaying the time series of images along with the at least one navigated surgical instrument used during the surgical procedure superimposed on the time series of images showing the real-time state of the imaged anatomy and the accurate positions of the at least one navigated surgical instrument within the real-time state of imaged anatomy.

2. The method of claim 1, wherein the patient's anatomy deforms in a predictable manner over time due to the ongoing body activity.

3. The method of claim 1, wherein the gating signal is a respiration cycle measurement signal of the patient.

4. The method of claim 1, wherein the gating signal is an EKG measurement signal of the patient.

5. The method of claim 1, wherein the step of acquiring a time series of images includes acquiring pre-operative or intraoperative images.

6. The method of claim 1, further comprising the step of continuously updating the display of the time series of images along with the navigated surgical instruments superimposed on the time series of images in sync with the gating signal.

7. A method of displaying real-time state of imaged anatomy during a surgical procedure comprising the steps of:
(a) attaching a measurement device to a patient to measure and ongoing body activity and generating a gating signal corresponding to the ongoing body activity;
(b) acquiring a time series of pre-operative 3D images of a patient's anatomy in a surgical field of interest in sync with the gating signal;
(c) establishing a navigation reference frame around the patient in the surgical field of interest;
(d) acquiring a time series of intraoperative 2D images of the patient's anatomy in the surgical field of interest in sync with the gating signal;
(e) reconstructing a time series of intraoperative 3D images from the time series of intraoperative 2D images;
(f) registering the time series of pre-operative 3D images with the time series of intraoperative 3D images; and
(g) displaying the time series of pre-operative 3D images along with at least one navigated surgical instrument used during the surgical procedure superimposed on the time series of pre-operative 3D images showing the real-time state of the imaged anatomy and the accurate positions of the at least one navigated surgical instrument within the real-time state of imaged anatomy.

8. The method of claim 7, wherein the patient's anatomy deforms in a predictable manner over time due to the ongoing body activity.

9. The method of claim 7, wherein the gating signal is a respiration cycle measurement signal of the patient.

10. The method of claim 7, wherein the gating signal is an EKG measurement signal of the patient.

11. The method of claim 7, wherein the step of reconstructing a time series of intraoperative 3D images from the time series of intraoperative 2D images includes determining an image position for each intraoperative 3D image in the time series in relation to the navigation reference frame.

12. The method of claim 7, wherein the step of registering the time series of pre-operative 3D images with the time series of intraoperative 3D images includes determining an image position for each pre-operative 3D image in the time series in relation to the navigation reference frame.

13. The method of claim 7, further comprising the step of determining a position for the at least one navigated surgical instrument used during the surgical procedure in relation to the navigation reference frame.

14. The method of claim 7, further comprising the step of continuously updating the display of the time series of images along with the navigated surgical instruments superimposed on the time series of images in sync with the gating signal.

15. A method of displaying real-time state of imaged anatomy during a surgical procedure comprising the steps of:
(a) attaching an EKG measurement device to a patient to measure the EKG of a patient and generating a gating signal corresponding to the measured EKG;
(b) acquiring a time sequence of pre-operative 3D CT volumetric images of a patient's anatomy in a surgical field of interest in sync with the gating signal;
(c) establishing a navigation reference frame around the patient in the surgical field of interest;
(d) acquiring a series of intraoperative 2D fluoroscopic images of the patient's anatomy in the surgical field of interest taken at various intervals while an imaging apparatus rotates around the patient;
(e) recording the EKG measurement and an image position within the navigation reference frame for each image in the series of intraoperative 2D fluoroscopic images;
(f) reconstructing a series of intraoperative 3D fluoroscopic volumetric images from the series of intraoperative 2D fluoroscopic images that have approximately the same EKG measurement;
(g) registering each intraoperative 3D fluoroscopic volumetric image in the time series with the navigation reference frame;
(h) registering each intraoperative 3D fluoroscopic volumetric image in the series with the corresponding pre-operative 3D CT volumetric image in the time sequence; and
(i) displaying the time sequence of pre-operative 3D CT volumetric images along with at least one navigated surgical instrument used during the surgical procedure superimposed on the time sequence of pre-operative 3D CT volumetric images showing the real-time state of the patient's imaged anatomy and the accurate positions of the at least one navigated surgical instrument within the real-time state of the patient's imaged anatomy.

16. The method of claim 15, wherein the patient's anatomy is a heart.

17. The method of claim 15, wherein the time sequence of pre-operative 3D CT volumetric images are acquired by a pre-operative 4D volumetric scan of the heart.

18. The method of claim 15, wherein the step of acquiring a time sequence of pre-operative 3D CT volumetric images includes recording and storing EKG time sequence measurements in sync with the time sequence of pre-operative 3D CT volumetric images, wherein a representative EKG measurement is determined for each pre-operative 3D CT volumetric image in the time sequence and stored with the time sequence.

19. The method of claim 15, wherein the series of intraoperative 2D fluoroscopic images are acquired by a fluoroscopic CT scan using a C-arm sweep where both the EKG measurement and the image position within the navigation reference frame are recorded and stored with each image in the sweep.

20. A method of displaying real-time state of imaged anatomy during a surgical procedure comprising the steps of:
(a) attaching a respiratory cycle measurement device to a patient to measure the respiratory cycle of the patient and generating a gating signal corresponding to the measured respiratory cycle;
(b) establishing a navigation reference frame around the patient in a surgical field of interest;
(c) acquiring a time series of 2D fluoroscopic images of a patient's anatomy in the surgical field of interest in sync with the gating signal;
(d) recording the respiratory cycle measurement and an image position within the navigation reference frame for each image in the time series of 2D fluoroscopic images; and
(e) displaying the time series of 2D fluoroscopic images along with navigated surgical instruments superimposed on the time series of 2D fluoroscopic images showing the real-time state of the patient's imaged anatomy and the accurate positions of the navigated surgical instruments within the real-time state of the patient's imaged anatomy.

21. The method of claim 20, wherein the time series of 2D fluoroscopic images are acquired by a fluoroscopic cine run from a non-moving fluoroscopic imaging apparatus.

22. The method of claim 20, wherein the patient's anatomy is a liver or other internal anatomy that deforms and changes position in sync with patient breathing.

23. The method of claim 20, wherein the fluoroscopic cine run is replayed with navigated surgical instruments accurately added to each 2D fluoroscopic image in the time series using the gating signal to drive the display sequence in real-time.

24. An image-guided surgery system comprising:
a measurement device coupled to a patient for measuring an ongoing body activity of the patient and generating a gating signal corresponding to the ongoing body activity measurement;
a plurality of tracking elements coupled to a navigation apparatus, wherein the navigation apparatus includes at least one processor;
at least one imaging apparatus coupled to the navigation apparatus configured for imaging a patient's anatomy that deforms and changes position in relation to the ongoing body activity; and
at least one display coupled to the a navigation apparatus and at least one imaging apparatus configured for displaying 2D slice and 3D volumetric images of the patient's anatomy that deforms and changes position in relation to the ongoing body activity and displaying an accurate position of at least one navigated surgical instrument within the 2D slice and 3D volumetric images in real-time.

25. The image-guided surgery system of claim 24, wherein the plurality of tracking elements includes at least one tracking elements attached to the at least one imaging apparatus, at least one attached to the at least one navigated surgical instrument, and at least one attached to or placed near the patient's anatomy that deforms and changes position in relation to the ongoing body activity,

26. The image-guided surgery system of claim 24, wherein the system synchronizes operation of the measurement device with the at least one imaging apparatus.

27. The image-guided surgery system of claim 24, wherein the at least one imaging apparatus includes a pre-operative imaging apparatus and an intraoperative imaging apparatus.
